SEQUENCE LISTING (1 sequence)

SEQ ID NO: 1 - protein, 585 aa, Homo Sapiens [sequence omitted]

(19) United States
(12) Patent Application Publication
(10) Pub. No.: US 2012/0148557 A1
(43) Pub. Date: Jun. 14, 2012

(54) ALBUMIN FUSED COAGULATION FACTORS FOR NON-INTRAVENOUS ADMINISTRATION IN THE THERAPY AND PROPHYLACTIC TREATMENT OF BLEEDING DISORDERS

(76) Inventor: Ulrich Kronthaler, Deisenhofen (DE)

(21) Appl. No.: 13/391,119

(22) PCT Filed: Aug. 18, 2010
(86) PCT No.: PCT/EP2010/062069

§ 371 Date: Feb. 17, 2012

(30) Foreign Application Priority Data
Aug. 20, 2009 (EP) 09010699.8

(51) Int. Cl. A61K 38/48; A61P 7/04; A61K 38/38

(52) U.S. Cl. 424/94.3; 514/13.7; 514/14.1

(57) ABSTRACT

The present invention relates to pharmaceutical preparations comprising albumin-fused coagulation factors for the non-intravenous administration in the therapy and prophylactic treatment of bleeding disorders and to a method for increasing the in-vivo recovery after non-intravenous administration of a coagulation factor by fusing it to albumin.

### FIELD OF THE INVENTION

The present invention relates to pharmaceutical preparations comprising albumin-fused coagulation factors for the non-intravenous administration in the therapy and prophylactic treatment of bleeding disorders and to a method for increasing the in-vivo recovery after non-intravenous administration of a coagulation factor by fusing it to albumin.

### BACKGROUND OF THE INVENTION

Several recombinant, therapeutic polypeptides are commercially available for therapeutic and prophylactic use in humans. The patients in general benefit from the specific mode of action of the recombinant active ingredients but currently all commercially available coagulation factors are administered via intravenous administration which often leads to infections at the injection site and is in general a procedure patients would like to avoid especially in the treatment of children with defects in their coagulation processes.

Ballance et al. (WO 01/79271) described fusion polypeptides of a multitude of different therapeutic polypeptides which, when fused to human serum albumin, are predicted to have an increased functional half-life in vivo and extended shelf-life. Long lists of potential fusion partners are described without showing by experimental data for almost any of these polypeptides that the respective albumin fusion proteins actually retain biological activity and have improved properties. Among the list of therapeutic polypeptides mentioned as examples are Factor IX and FVII/FVIIa. Also described are fusions of FIX and FVII/FVIIa in which there is a peptide linker between albumin and FIX or FVII/FVIIa. However, there is no disclosure that an albumin fusion could improve therapeutic treatments when the respective coagulation factor is administered non-intravenously.

### Factor VII and Factor VIIa

FVII is a single-chain glycoprotein with a molecular weight of 50 kDa, which is secreted by liver cells into the blood stream as an inactive zymogen of 406 amino acids. FVII is converted to its active form Factor VIIa, by proteolysis of the single peptide bond at Arg152-IIe153 leading to the formation of two polypeptide chains, a N-terminal light chain (24 kDa) and a C-terminal heavy chain (28 kDa), which are held together by one disulfide bridge. In contrast to other vitamin K-dependent coagulation factors, no activation peptide is cleaved off during activation. Activation cleavage of Factor VII can be achieved in vitro, for example, by Factor Xa, Factor IXa, Factor VIIa, Factor XIIa, Factor Seven Activating Protease (FSAP), and thrombin. Mollerup et al. (Biotechnol. Bioeng. (1995) 48: 501-505) reported that some cleavage also occurs in the heavy chain at Arg290 and/or Arg315.

Factor VII is present in plasma in a concentration of 500 ng/ml. About 1% or 5 ng/ml of Factor VII is present as activated Factor VIIa. The terminal plasma half-life of Factor VII was found to be about 4 hours and that of Factor VIIa about 2 hours.

By administering supraphysiological concentrations of Factor VIIa hemostasis can be achieved bypassing the need for Factor VIIIa and Factor IXa. The cloning of the cDNA for Factor VII (U.S. Pat. No. 4,784,950) made it possible to develop activated Factor VII as a pharmaceutical. Factor VIIa was successfully administered for the first time in 1988. Ever since the number of indications of Factor VIIa has grown steadily showing a potential to become an universal hemostatic agent to stop bleeding (Erhardtsen, 2002). However, the short terminal half-life of Factor VIIa of approximately 2 hours and reduced in vivo recovery is limiting its application.

### Human FIX

Human FIX, one member of the group of vitamin K-dependent polypeptides, is a single-chain glycoprotein with a molecular weight of 57 kDa, which is secreted by liver cells into the blood stream as an inactive zymogen of 415 amino acids. It contains 12 y-carboxy-glutamic acid residues localized in the N-terminal Gla-domain of the polypeptide. The Gla residues require vitamin K for their biosynthesis. Following the Gla domain there are two epidermal growth factor domains, an activation peptide, and a trypsin-type serine protease domain. Further posttranslational modifications of FIX encompass hydroxylation (Asp 64), N-(Asn157 and Asn167) as well as O-type glycosylation (Ser53, Ser61, Thr159, Thr169, and Thr172), sulfation (Tyr155), and phosphorylation (Ser158).

FIX is converted to its active form, Factor IXa, by proteolysis of the activation peptide at Arg145-Ala146 and Arg180-Val181 leading to the formation of two polypeptide chains, an N-terminal light chain (18 kDa) and a C-terminal heavy chain (28 kDa), which are held together by one disulfide bridge. Activation cleavage of Factor IX can be achieved in vitro e.g. by Factor XIa or Factor VIIa/TF. Factor IX is present in human plasma in a concentration of 5-10 μg/ml. Terminal plasma half-life of Factor IX in humans was found to be about 15 to 18 hours (White G C et al. 1997. Recombinant factor IX. Thromb Haemost. 78: 261-265; Ewenstein B M et al. 2002. Pharmacokinetic analysis of plasma-derived and recombinant F IX concentrates in previously treated patients with moderate or severe hemophilia B. Transfusion 42:190-197).

Hemophilia B is caused by non-functional or missing Factor IX and is treated with Factor IX concentrates from plasma or a recombinant form of Factor IX. As haemophilia B patients often receive at least biweekly prophylactic administrations of Factor IX to avoid spontaneous bleedings, it is desirable to increase the intervals of between administration by increasing the half-life of the Factor IX product applied and it is desirable to avoid the intravenous administration of Factor IX.

### Factor VIII (FVIII)

FVIII is a blood plasma glycoprotein of about 260 kDa molecular mass, produced in the liver of mammals. It is a critical component of the cascade of coagulation reactions that lead to blood clotting. Within this cascade is a step in which factor IXa (FIXa), in conjunction with FVIII, converts factor X (FX) to an activated form, FXa. FVIII acts as a cofactor at this step, being required with calcium ions and phospholipid for the activity of FIXa. The most common hemophilic disorders is caused by a deficiency of functional FVIII called hemophilia A.

An important advance in the treatment of Hemophilia A has been the isolation of cDNA clones encoding the complete 2,351 amino acid sequence of human FVIII (U.S. Pat. No. 4,757,006) and the provision of the human FVIII gene DNA sequence and recombinant methods for its production).

Analysis of the deduced primary amino acid sequence of human FVIII determined from the cloned cDNA indicates that it is a heterodimer processed from a larger precursor polypeptide. The heterodimer consists of a C-terminal light chain of about 80 kDa in a metal ion-dependent association with an about 210 kDa N-terminal heavy chain fragment. (See review by Kaufman, Transfusion Med. Revs. 6:235 (1992)). Physiological activation of the heterodimer occurs through proteolytic cleavage of the protein chains by thrombin. Thrombin cleaves the heavy chain to a 90 kDa protein, and then to 54 kDa and 44 kDa fragments. Thrombin also cleaves the 80 kDa light chain to a 72 kDa protein. It is the latter protein, and the two heavy chain fragments (54 kDa and 44 kDa above), held together by calcium ions, that constitute active FVIII. Inactivation occurs when the 72 kDa and 54 kDa proteins are further cleaved by thrombin, activated protein C or FXa. In plasma, this FVIII complex is stabilized by association with a 50-fold excess of VWF protein (“VWF”), which appears to inhibit proteolytic destruction of FVIII as described above.

The amino acid sequence of FVIII is organized into three structural domains: a triplicated A domain of 330 amino acids, a single B domain of 980 amino acids, and a duplicated C domain of 150 amino acids. The B domain has no homology to other proteins and provides 18 of the 25 potential asparagine(N)-linked glycosylation sites of this protein. The B domain has apparently no function in coagulation and can be deleted with the B-domain deleted FVIII molecule still having procoagulatory activity.

Von Willebrand Factor (vWF)

VWF is a multimeric adhesive glycoprotein present in the plasma of mammals, which has multiple physiological functions. During primary hemostasis VWF acts as a mediator between specific receptors on the platelet surface and components of the extracellular matrix such as collagen. Moreover, VWF serves as a carrier and stabilizing protein for procoagulant FVIII. VWF is synthesized in endothelial cells and megakaryocytes as a 2813 amino acid precursor molecule. The precursor polypeptide, pre-pro-VWF, consists of a 22-residue signal peptide, a 741-residue pro-peptide and the 2050-residue polypeptide found in mature plasma VWF (Fischer et al., FEBS Lett. 351: 345-348, 1994). Upon secretion into plasma VWF circulates in the form of various species with different molecular sizes. These VWF molecules consist of oligo- and multimers of the mature subunit of 2050 amino acid residues. VWF can be usually found in plasma as one dimer up to multimers consisting of 50-100 dimers (Ruggeri et al. Thromb. Haemost. 82: 576-584, 1999). The in vivo half-life of human VWF in the human circulation is approximately 12 to 20 hours.

The most frequent inherited bleeding disorder in humans is von Willebrand's disease (VWD), which can be treated by replacement therapy with concentrates containing VWF of plasmatic or recombinant origin.

VWF can be prepared from human plasma as for example described in EP 05503991. In patent EP 0784632 a method for isolating recombinant VWF is described.

VWF is known to stabilize FVIII in vivo and, thus, plays a crucial role to regulate plasma levels of FVIII and as a consequence is a central factor to control primary and secondary hemostasis. It is also known that after intravenous administration pharmaceutical preparations containing VWF in VWD patients an increase in endogenous FVIII:C to 1 to 3 units per ml in 24 hours can be observed demonstrating the in vivo stabilizing effect of VWF on FVIII.

Until today the standard treatment of Hemophilia A and VWD involves frequent intravenous infusions of preparations of FVIII and VWF concentrates. The treatment of Hemophilia B requires the biweekly administration of Factor IX and in the treatment of inhibitor patients with FVIIa, multiple administrations of FVIIa per week are used to avoid bleedings.

These replacement therapies are generally effective, however, for example in severe hemophilia A patients undergoing prophylactic treatment Factor VIII has to be administered intravenously (i.v.) about 3 times per week due to the short plasma half life of Factor VIII of about 12 hours. Already above levels of 1% of the FVIII activity in non-hemophiliacs, e.g. by a raise of FVIII levels by 0.01 U/ml, severe hemophilia A is turned into moderate hemophilia A. In prophylactic therapy dosing regimes are designed such that the trough levels of FVIII activity do not fall below levels of 2-3% of the FVIII activity in non-hemophiliacs.

The administration of a coagulation factor via intravenous administration is cumbersome, associated with pain and entails the risk of an infection especially as this is mostly done in home treatment by the patients themselves or by the parents of children being diagnosed for hemophilia A. In addition frequent intravenous injections inevitably result in scar formation, interfering with future infusions As prophylactic treatment in severe hemophilia is started early in life, with children often being less than 2 years old, it is even more difficult to inject FVIII 3 times per week into the veins of such small patients. For a limited period, implantation of port systems may offer an alternative. Despite the fact that repeated infections may occur and ports can cause inconvenience during physical exercise, they are nevertheless typically considered as favourable as compared to intravenous injections.

Thus there is a great medical need to obviate the need to infuse coagulation factors intravenously.

### DESCRIPTION OF THE INVENTION

The present invention relates to pharmaceutical preparations comprising albumin-fused coagulation factors for the non-intravenous administration in the therapy and prophylactic treatment of bleeding disorders and to a method for increasing the in-vivo recovery after non-intravenous administration of a coagulation factor by fusing it to albumin.

Preferred coagulation factors are vitamin-K dependent coagulation factors and fragments and variants thereof. Even more preferred are FVIIa and FIX and fragments and variants thereof. Also preferred without limitation are albumin-fused FVIII and vWF, fibrinogen, factor II, factor X, factor XIII, thrombin, prothrombin and protein C.

Preferably the formulation comprising albumin-fused coagulation factors is administered subcutaneously. However all other modes of non-intravenous administration are encompassed, e.g. intramuscular or intradermal administration.

The coagulation factor may be fused to albumin via a peptidic linker which may be cleavable by proteases in certain embodiments of the invention.

The gist of the invention is demonstrated in particular by the vitamin K-dependent polypeptide Factor VII and albumin. The invention also relates to other coagulation factors. The invention also related to cDNAs coding for albumin-fused coagulation factors. The cDNA coding for the respective coagulation factor are genetically fused to cDNA sequences coding for human serum albumin and may be linked by oligonucleotides that code for intervening peptide linkers which may be cleavable by proteases. The invention also relates to the use of recombinant expression vectors containing such fused cDNA sequences for non-intravenous administration. This could be for example gene therapy via intramuscular injection of expression vectors comprising a cDNA coding for an albumin-fused coagulation factor.

### DETAILED DESCRIPTION OF THE INVENTION

“Albumin-fused coagulation factor” in the sense of the invention means a genetic fusion of human albumin with a coagulation factor in which the plasma half-life of the resulting fusion polypeptide is increased in comparison to the same but non-fused coagulation factor and in which the in vivo recovery after non-intravenous administration is increased as compared to the in vivo recovery after non-intravenous administration of the same but non-fused coagulation factor.

Preferred embodiments of the invention are albumin-fused coagulation factors in which the in vivo recovery after non-intravenous administration is increased by at least 10%, preferable at least 25%, more preferably more than 50%, more preferably more than 100% and even more preferably more than 200% as compared to the in vivo recovery after non-intravenous administration of the same but non-fused coagulation factor.

“In vivo recovery after non-intravenous administration” means the amount of biologically active coagulation factor which can be found in plasma after non-intravenous administration.

The in vivo recovery after non-intravenous administration can be determined for example as “area under the curve”, or as “peak plasma level” by using analytical coagulation-related assays for determining the biological activity of the respective coagulation factor which are well know in the art. “In vivo recovery” in the sense of the invention means the amount of product found in blood or plasma shortly after administration of the product. Therefore, for detection of the in vivo recovery in general the plasma content is determined for example 15 min, or 60 min or 2 h or 4 h or 8 h or 12 h or 20 h after administration of the product.

“Coagulation-related assays” in the sense of the invention is any assay which determines enzymatic or cofactor activities that are of relevance in the coagulation process or that is able to determine that either the intrinsic or the extrinsic coagulation cascade has been activated. The “coagulation-related” assay thus may be direct coagulation assays like aPTT, PT, or the thrombin generation assays. However, other assays like, e.g., chromogenic assays applied for specific coagulation factors are also included. Examples for such assays or corresponding reagents are Pathromtin® SL (aPTT assay, Dade Behring) or Thromborel® S (Prothrombin time assay, Dade Behring) with corresponding coagulation factor deficient plasma (Dade Behring), Thrombin generation assay kits (Technoclone, Thrombinoscope) using e.g. coagulation factor deficient plasma, chromogenic assays like Biophen Factor IX (Hyphen BioMed), Staclot® FVIIa-rTF (Roche Diagnostics GmbH), Coatest® Factor VIII:C/4 (Chromogenix), or others.

As a surrogate for determining biologically active coagulations factors also the amount of antigen of the respective coagulation factor can be determined. Antigen levels are preferably determined by techniques like ELISA testing.

“Coagulation factor” in the sense of the invention is any polypeptide which can contribute to primary or secondary hemostasis. “Coagulation factors” include, but are not limited to, polypeptides consisting of Factor IX, Factor VII, Factor VIII, von Willebrand Factor, Factor V, Factor X, Factor XI, Factor XII, Factor XIII, Factor I, Factor II (Prothrombin), Protein C, Protein S, GAS6, or Protein Z as well as their activated forms. Furthermore, useful coagulation factors may be wild-type polypeptides or may contain mutations. The degree and location of glycosylation or other post-translation modifications may vary depending on the chosen host cells and the nature of the host cellular environment. When referring to specific amino acid sequences, posttranslational modifications of such sequences are encompassed in this application.

### Albumin

As used herein, “albumin” refers collectively to albumin polypeptide or amino acid sequence, or an albumin fragment or variant having one or more functional activities (e.g., biological activities) of albumin. In particular, “albumin” refers to human albumin or fragments thereof, especially the mature form of human albumin as shown in SEQ ID No:1 herein or albumin from other vertebrates or fragments thereof, or analogs or variants of these molecules or fragments thereof.

The albumin portion of the albumin fusion proteins may comprise the full length of the HA sequence as described above, or may include one or more fragments thereof that are capable of stabilizing or prolonging the therapeutic activity. Such fragments may be of 10 or more amino acids in length or may include about 15, 20, 25, 30, 50, or more contiguous amino acids from the HA sequence or may include part or all of specific domains of HA.

The albumin portion of the albumin fusion proteins of the invention may be a variant of normal HA, either natural or artificial. The therapeutic polypeptide portion of the fusion proteins of the invention may also be variants of the corresponding therapeutic polypeptides as described herein. The term “variants” includes insertions, deletions, and substitutions, either conservative or non-conservative, either natural or artificial, where such changes do not substantially alter the active site, or active domain that confers the therapeutic activities of the therapeutic polypeptides.

In particular, the albumin fusion proteins of the invention may include naturally occurring polymorphic variants of human albumin and fragments of human albumin. The albumin may be derived from any vertebrate, especially any mammal, for example human, cow, sheep, or pig. Non-mammalian albumins include, but are not limited to, hen and salmon. The albumin portion of the albumin-linked polypeptide may be from a different animal than the therapeutic polypeptide portion.

Generally speaking, an albumin fragment or variant will be at least 10, preferably at least 40, most preferably more than 70 amino acids long. The albumin variant may preferentially consist of or alternatively comprise at least one whole domain of albumin or fragments of said domains, for example domains 1 (amino acids 1-194 of SEQ ID NO:1), 2 (amino acids 195-387 of SEQ ID NO: 1), 3 (amino acids 388-585 of SEQ ID NO: 1), 1+2 (1-387 of SEQ ID NO: 1), 2+3 (195-585 of SEQ ID NO: 1) or 1+3 (amino acids 1-194 of SEQ ID NO: 1+amino acids 388-585 of SEQ ID NO: 1). Each domain is itself made up of two homologous subdomains namely 1-105, 120-194, 195-291, 316-387, 388-491 and 512-585, with flexible inter-subdomain linker regions comprising residues Lys106 to Glu119, Glu292 to Val315, and Glu492 to Ala511.

The albumin portion of an albumin fusion protein of the invention may comprise at least one subdomain or domain of HA or conservative modifications thereof.

All fragments and variants of albumin are encompassed by the invention as fusion partners of a coagulation factor as long as they lead to a half-life extension of the therapeutic fusion protein in plasma of at least 25% as compared to the non-fused coagulation factor.

“Albumin-fused coagulation factor” as used in this application means a polypeptide comprising the nonactivated as well as the activated forms of the respective coagulation factor. “Albumin-fused coagulation factor” as used in this invention include proteins that have the amino acid sequence of native coagulation factor and of albumin respectively. It also includes proteins with a slightly modified amino acid sequence, for instance, a modified N-terminal end including N-terminal amino acid deletions or additions so long as those proteins substantially retain the biological activity of the respective coagulation factor. “Albumin-fused coagulation factors” within the above definition also include natural allelic variations that may exist and occur from one individual to another. “Albumin-fused coagulation factors” within the above definition further include variants of the respective coagulation factors and of albumin. Such variants differ in one or more amino acid residues from the wild type sequence. Examples of such differences may include truncation of the N- and/or C-terminus by one or more amino acid residues (e.g. 1 to 10 amino acid residues), or addition of one or more extra residues at the N- and/or C-terminus, e.g. addition of a methionine residue at the N-terminus, as well as conservative amino acid substitutions, i.e. substitutions performed within groups of amino acids with similar characteristics, e.g. (1) small amino acids, (2) acidic amino acids, (3) polar amino acids, (4) basic amino acids, (5) hydrophobic amino acids, (6) aromatic amino acids. Examples of such conservative substitutions are shown in the following table.

TABLE 1

| | | | | |
|---|---|---|---|---|
| (1) | Alanine | Glycine | | |
| (2) | Aspartic acid | Glutamic acid | | |
| (3a) | Asparagine | Glutamine | | |
| (3b) | Serine | Threonine | | |
| (4) | Arginine | Histidine | Lysine | |
| (5) | Isoleucine | Leucine | Methionine | Valine |
| (6) | Phenylalanine | Tyrosine | Tryptophane | |

The in vivo half-life of the fusion proteins of the invention, in general determined as terminal half-life or 13-half-life, is usually at least about 25%, preferable at least about 50%, and more preferably more than 100% higher than the in vivo half-life of the non-fused polypeptide.

The linker region in a preferred embodiment comprises a sequence of the coagulation factor to be administered or a variant thereof, which should result in a decreased risk of neoantigenic properties (formation of a novel potentially immunogenic epitope due to the occurrence of a peptide within the therapeutic antigen which does not exist in human proteins) of the expressed fusion protein. Also in case the coagulation factor is a zymogen (e.g. needs to be proteolytically activated) the kinetics of the peptide linker cleavage will more closely reflect the coagulation-related activation kinetics of the zymogen. Thus, in such preferred embodiments a zymogen and a corresponding linker are activated and respectively cleaved, with comparable kinetics. For this reason, the present invention also particularly relates to fusion proteins of a zymogen and albumin.

In a further embodiment, the linker peptide comprises cleavage sites for more than one protease. This can be achieved either by a linker peptide that can be cleaved at the same position by different proteases or by a linker peptide that provides two or more different cleavage sites. This may be advantageous circumstances where the therapeutic fusion protein must be activated by proteolytic cleavage to achieve enzymatic activity and where different proteases may contribute to this activation step. This is the case, for example, upon activation of FIX, which can either be achieved by FXIa or by FVIIa/Tissue Factor (TF).

Preferred embodiments of the invention are therapeutic fusion proteins wherein the linker is cleavable by the protease, that activates the coagulation factor, thereby ensuring that the cleavage of the linker is linked to the activation of the coagulation factor at a site at which coagulation occurs.

Other preferred therapeutic fusion proteins according to the invention are those, wherein the linker is cleavable by the coagulation factor which is part of the therapeutic fusion protein once it is activated, thus also ensuring that cleavage of the fusion protein is connected with a coagulatory event.

Other preferred therapeutic fusion proteins according to the invention are those, wherein the linker is cleavable by a protease, which itself is activated directly or indirectly by the activity of the coagulation factor which is part of the therapeutic fusion protein, thus also ensuring that cleavage of the fusion protein is connected with a coagulatory event.

One class of most preferred therapeutic fusion proteins are those wherein the linker is cleavable by FXIa and/or by FVIIa/TF and the coagulation factor is FIX

Another embodiment of the invention is the use of a pharmaceutical composition comprising albumin-fused coagulation factors for non-intravenous administration. The mode of administration is preferentially subcutaneous, but encompasses all extravascular routes of administration. Encompassed is also administration via epithelial surfaces (e.g. on the skin). Of special clinical utility would be an application via a patch. This topical administration requires uptake through the skin, which can be however quite marked, not only with superficial abrasions but also intact skin, and it may include eye drops and nasal applications. Administration via epithelial surfaces includes inhalation, which is suitable due to the extraordinary large surface covered with the protein, leading to rapid uptake and bypassing of the liver. Administration on epithelial surfaces includes dosage forms which are held in the mouth or under the tongue, i.e. are buccal or sublingual dosage forms, possibly even as chewing gum. Since the pH in the mouth is relatively neutral (as opposed to the acidic stomach milieu) this would be positive for a labile protein such as FVIII. Vaginal and even rectal administration might also be considered as some of the veins draining the rectum lead directly to the general circulation. Typically this is most helpful for patients who cannot take substances via the oral route, such as young children.

Intradermal injection (in the skin) would be a more invasive mode of administration, but still suitable for a treatment without assistance or even execution by trained personnel. Intradermal administration would be followed by subcutaneous injection (just under the skin). Typically uptake is quite substantial and can be increased by warming or massaging the injection area. Alternatively vasoconstriction can be achieved, resulting in the opposite behaviour, i.e. reducing the adsorption but prolonging the effect.

Even more invasive extravascular administration includes intramuscular delivery (into the body of the muscle). This might provide benefits by circumventing adipose tissue, but it is typically more painful that subcutaneous injections and especially with patients characterized by a deficient coagulation system, to be improved by the injection, there is the risk of tissue lesions, resulting in bleedings.

The coagulation factors of the present invention are administered to patients in a therapeutically effective dose, meaning a dose that is sufficient to produce the desired effects, preventing or lessening the severity or spread of the condition or indication being treated without reaching a dose which produces intolerable adverse side effects. The exact dose depends on many factors as e.g. the indication, formulation, mode of administration and has to be determined in preclinical and clinical trials for each respective indication.

The coagulation factors of the present invention can be used to treat bleedings in familial and in acquired cases of hemophilia A and B, familial or acquired von Willebrand disease, all types of trauma, (blunt or penetrating, leading to severe hemorrhage either from a single organ, a bone fraction or from polytrauma, bleeding during surgical procedures including peri- or postoperative haemorrhage, bleeding due to cardiac surgery including patients undergoing extracorporal circulation and hemodilution in pediatric cardiac surgery, intracerebral hemorrhage, subarachnoid hemorrhage, sub- or epidural bleeding, bleedings due to blood loss and hemodilution, by non-plasmatic volume substitution leading to reduced levels of coagulation factors in affected patients, bleedings due to disseminated intravascular coagulation (DIC) and a consumption coagulopathy, thrombocyte dysfunctions, depletion and coagulopathies, bleeding due to liver cirrhosis, liver dysfunction and fulminant liver failure, liver biopsy in patients with liver disease, bleeding after liver and other organ transplantations, bleeding from gastric varices and peptic ulcer bleeding, gynaecological bleedings as dysfunctional uterine bleeding (DUB), premature detachment of the placenta, periventricular haemorrhage in low birth weight children, post partum haemorrhage, fatal distress of newborns, bleeding associated with burns, bleeding associated with amyloidosis, hematopoietic stem cell transplantation associated with platelet disorder, bleedings associated with malignancies, infections with haemorrhaging viruses, bleeding associated with pancreatitis.

The invention further relates to the use of polynucleotides encoding albumin-fused coagulation factors as described in this application. The term “polynucleotide(s)” generally refers to any polyribonucleotide or polydeoxyribonucleotide that may be unmodified RNA or DNA or modified RNA or DNA. The polynucleotide may be single- or double-stranded DNA, single or double-stranded RNA. As used herein, the term “polynucleotide(s)” also includes DNAs or RNAs that comprise one or more modified bases and/or unusual bases, such as inosine. It will be appreciated that a variety of modifications may be made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells.

The skilled person will understand that, due to the degeneracy of the genetic code, a given polypeptide can be encoded by different polynucleotides. These “variants” are encompassed by this invention.

Preferably, the polynucleotide of the invention is a purified polynucleotide. The term “purified” polynucleotide refers to a polynucleotide that is substantially free from other nucleic acid sequences, such as and not limited to other chromosomal and extra-chromosomal DNA and RNA. Purified polynucleotides may be purified from a host cell. Conventional nucleic acid purification methods known to skilled artisans may be used to obtain purified polynucleotides. The term also includes recombinant polynucleotides and chemically synthesized polynucleotides.

Yet another aspect of the invention is the use of a plasmid or vector comprising a polynucleotide according to the invention. Preferably, the plasmid or vector is an expression vector. In a particular embodiment, the vector is a transfer vector for use in human gene therapy.

Degree and location of glycosylation or other post-translation modifications may vary depending on the chosen host cells and the nature of the host cellular environment. When referring to specific amino acid sequences, posttranslational modifications of such sequences are encompassed in this application.

The term “recombinant” means, for example, that the variant has been produced in a host organism by genetic engineering techniques. The FVIII or VWF variant of this invention is usually a recombinant variant.

### Expression of the Proposed Variants:

The production of recombinant proteins at high levels in suitable host cells, requires the assembly of the above-mentioned modified cDNAs into efficient transcriptional units together with suitable regulatory elements in a recombinant expression vector, that can be propagated in various expression systems according to methods known to those skilled in the art. Efficient transcriptional regulatory elements could be derived from viruses having animal cells as their natural hosts or from the chromosomal DNA of animal cells. Preferably, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 can be used. In order to achieve stable high levels of mRNA transcribed from the cDNAs, the transcriptional unit should contain in its 3′-proximal part a DNA region encoding a transcriptional termination-polyadenylation sequence. Preferably, this sequence is derived from the Simian Virus 40 early transcriptional region, the rabbit beta-globin gene, or the human tissue plasminogen activator gene.

The cDNAs are then integrated into the genome of a suitable host cell line for expression of the albumin-fused coagulation factors of the invention. Preferably this cell line should be an animal cell-line of vertebrate origin in order to ensure correct folding, Gla-domain synthesis, disulfide bond formation, asparagine-linked glycosylation, O-linked glycosylation, and other post-translational modifications as well as secretion into the cultivation medium. Examples of other post-translational modifications are hydroxylation and proteolytic processing of the nascent polypeptide chain. Examples of cell lines that can be used are monkey COS-cells, mouse L-cells, mouse C127-cells, hamster BHK-21 cells, human embryonic kidney 293 cells, and hamster CHO-cells.

The recombinant expression vector encoding the corresponding cDNAs can be introduced into an animal cell line in several different ways. For instance, recombinant expression vectors can be created from vectors based on different animal viruses. Examples of these are vectors based on baculovirus, vaccinia virus, adenovirus, and preferably bovine papilloma virus.

The transcription units encoding the corresponding DNAs can also be introduced into animal cells together with another recombinant gene, which may function as a dominant selectable marker in these cells in order to facilitate the isolation of specific cell clones, which have integrated the recombinant DNA into their genome. Examples of this type of dominant selectable marker genes are Tn5 amino glycoside phosphotransferase, conferring resistance to geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the cDNA of the desired protein, or it can be encoded on a separate vector which is simultaneously introduced and integrated into the genome of the host cell, frequently resulting in a tight physical linkage between the different transcription units.

Other types of selectable marker genes, which can be used together with the cDNA of the desired protein, are based on various transcription units encoding dihydrofolate reductase (dhfr). After introduction of this type of gene into cells lacking endogenous dhfr-activity, preferentially CHO-cells (DUKX-B11, DG-44) it will enable these to grow in media lacking nucleosides. An example of such a medium is Ham's F12 without hypoxanthine, thymidine, and glycine. These dhfr-genes can be introduced together with the coagulation factor cDNA transcriptional units into CHO-cells of the above type, either linked on the same vector or on different vectors, thus creating dhfr-positive cell lines producing recombinant protein.

If the above cell lines are grown in the presence of the cytotoxic dhfr-inhibitor methotrexate, new cell lines resistant to methotrexate will emerge. These cell lines may produce recombinant protein at an increased rate due to the amplified number of linked dhfr and the desired protein's transcriptional units. When propagating these cell lines in increasing concentrations of methotrexate (1-10000 nM), new cell lines can be obtained which produce the desired protein at very high rate.

The above cell lines producing the desired protein can be grown on a large scale, either in suspension culture or on various solid supports. Examples of these supports are micro carriers based on dextran or collagen matrices, or solid supports in the form of hollow fibres or various ceramic materials. When grown in cell suspension culture or on micro carriers the culture of the above cell lines can be performed either as a bath culture or as a perfusion culture with continuous production of conditioned medium over extended periods of time. Thus, according to the present invention, the above cell lines are well suited for the development of an industrial process for the production of the desired recombinant proteins. The recombinant protein, which accumulates in the medium of secreting cells of the above types, can be concentrated and purified by a variety of biochemical and chromatographic methods, including methods utilizing differences in size, charge, hydrophobicity, solubility, specific affinity, etc. between the desired protein and other substances in the cell cultivation medium.

An example of such purification is the adsorption of the recombinant protein to a monoclonal antibody, which is immobilised on a solid support. After desorption, the protein can be further purified by a variety of chromatographic techniques based on the above properties.

It is preferred to purify the modified biologically active albumin-fused coagulation factor of the present invention to 80% purity, more preferably 95% purity, and particularly preferred is a pharmaceutically pure state that is greater than 99.9% pure with respect to contaminating macromolecules, particularly other proteins and nucleic acids, and free of infectious and pyrogenic agents. Preferably, an isolated or purified modified biologically active albumin-fused coagulation factor of the invention is substantially free of other polypeptides except when a combination with other therapeutic proteins is to be administered.

The albumin-fused coagulation factors described in this invention can be formulated into pharmaceutical preparations for therapeutic use. The purified proteins may be dissolved in conventional physiologically compatible aqueous buffer solutions to which there may be added, optionally, pharmaceutical excipients to provide pharmaceutical preparations.

Such pharmaceutical carriers and excipients as well as suitable pharmaceutical formulations are well known in the art (see for example “Pharmaceutical Formulation Development of Peptides and Proteins”, Frokjaer et al., Taylor & Francis (2000) or “Handbook of Pharmaceutical Excipients”, 3^{rd} edition, Kibbe et al., Pharmaceutical Press (2000)). In particular, the pharmaceutical composition comprising the polypeptide variant of the invention may be formulated in lyophilized or stable soluble form. The polypeptide variant may be lyophilized by a variety of procedures known in the art. Lyophilized formulations are reconstituted prior to use by the addition of one or more pharmaceutically acceptable diluents such as sterile water for injection or sterile physiological saline solution.

Formulations of the composition are delivered to the individual by any pharmaceutically suitable means of non-intravenous administration. Various delivery systems are known and can be used to administer the composition by any convenient route. Preferentially the compositions of the invention are formulated for subcutaneous, intramuscular, intraperitoneal, intracerebral, intrapulmonar, intranasal or transdermal administration, most preferably for subcutaneous, intramuscular or transdermal administration according to conventional methods. The formulations can be administered continuously by infusion or by bolus injection. Some formulations encompass slow release systems.

The albumin-fused coagulation factors of the present invention are administered to patients in a therapeutically effective dose, meaning a dose that is sufficient to produce the desired effects, preventing or lessening the severity or spread of the condition or indication being treated without reaching a dose which produces intolerable adverse side effects. The exact dose depends on many factors as e.g. the indication, formulation, mode of administration and has to be determined in preclinical and clinical trials for each respective indication.

The pharmaceutical composition of the invention may be administered alone or in conjunction with other therapeutic agents. These agents may be incorporated as part of the same pharmaceutical.

### FIGURES

FIG. 1: Time course of FVII:Ag plasma concentration following 300 μg/kg rVIIa-FP and NovoSeven® (pool; n=3/timepoint; linear scale)

FIG. 2: Time course of FVII:Ag plasma concentration following 300 μg/kg rVIIa-FP and NovoSeven® (pool; n=3/timepoint; log-linear scale)

FIG. 3: Time course of FIX:Ag plasma concentration following 610 μg/kg rIX-FP and Berinin® (pool; n=5/timepoint; linear scale)

FIG. 4: Time course of FIX:Ag plasma concentration following 610 μg/kg rIX-FP and Berinin® (pool; n=5/timepoint; log-linear scale)

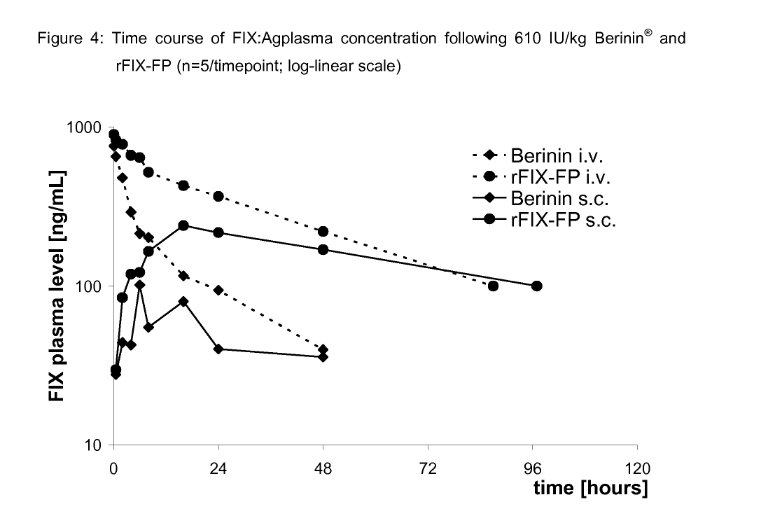

### EXAMPLES

### Example 1

### Assessment of Bioavailability of s.c. Applied rVIIa-FP in Rats

The goal of the experiments summarized in the first example was to assess, whether extravascular injections might be an option for an improved therapy with rVIIa-FP (human). As a typical representative for an extravascular therapy, subcutaneous injection was chosen. In order to compare the suitability of rVIIa-FP to the reference product NovoSeven®, a non-clinical pharmacokinetic study with a design detailed in Table 2 was performed. The time course of plasma levels was determined following a single intravenous/subcutaneous injection of equimolar doses of rVIIa-FP (pFVII-937 produced as described in WO 2007/090584 on page 30 to 31) and NovoSeven® to rats. For NovoSeven® the dose was 300 μg/kg. The dose of the rVIIa-FP was based on the protein concentration, as determined by OD measurement (280-320 nm). Corrected for the albumin part of the FP, a dose of 300 μg (FVIIa)/kg, was applied as well. By this, both products were applied at the same dose with regard to the therapeutically efficient component, FVIIa. Rats were selected as animal species for this study, because the represent a well characterized and frequently used species for this type of studies. Rats (CD strain) were provided by Charles River (Sulzfeld, Germany), weighing about 200 g during the experiment. Rats were kept at standard housing conditions. Under short term anesthesia, blood samples were drawn retroorbitally, anticoagulated using calcium citrate to 10 to 20% citrate blood, processed to plasma and stored at −20° C. for the determination of FVII plasma level. Sampling timepoints are detailed in table 2. Plasma concentrations of human FVII were determined using a sandwich ELISA, using sheep anti-FVII IgG (Cedarlane/Biozol) as capture antibody and POD conjugated sheep anti-FVII IgG (Cedarlane/Biozol) as detection antibody. Standard human plasma was used as reference. Fermentation, purification and activation of rVIIa-FP have been described elsewhere.

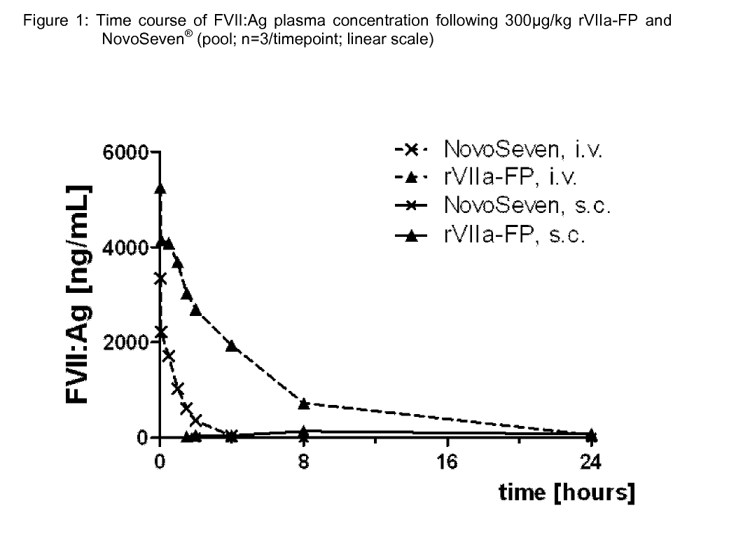

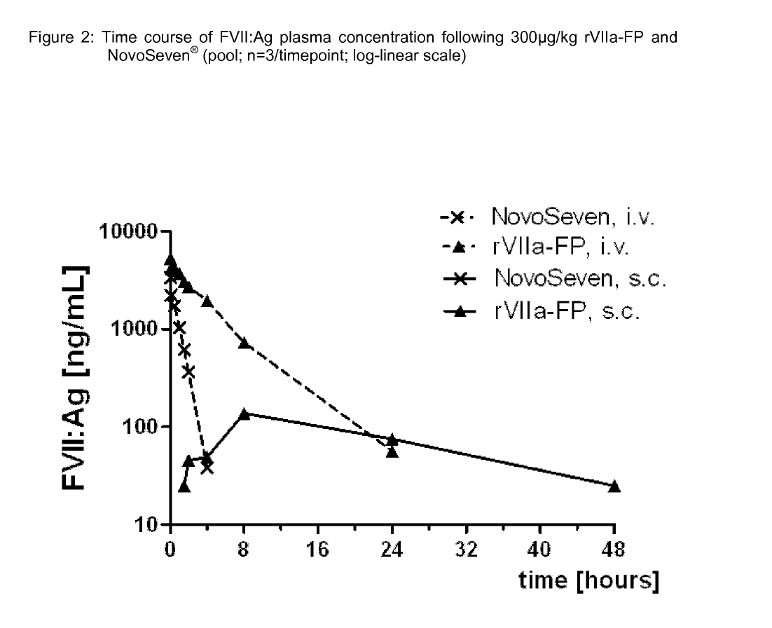

Intravenous injection of rVIIa-FP resulted in an initial plasma level, which was about 60% higher as compared to the treatment with the same dose NovoSeven® (Table 3, FIG. 1). Only for the group treated subcutaneously with rVIIa-FP, FVII was detected in plasma. The maximal concentration was observed at about 8 hours following injection and baseline was reached again 1 to 2 days following treatment. For the group treated subcutaneously with the same dose NovoSeven®, plasma levels remained below the detection limit during the entire observation period (FIG. 2), although NovoSeven® was injected at the same FVIIa dose as rVIIa-FP.

TABLE 2
Treatment groups

| | | FVIIa Dose | volume | | | N |
|---|---|---|---|---|---|---|
| No. | Treatment | [μg/kg] | [mL/kg] | schedule | route | (M/F) |
| 1 | NovoSeven ® | 300 | 4.0 | single | i.v. | 0/6 |
| | | | | injection | | |
| | | | | (t = 0) | | |
| 2 | rVIIa-FP | 300 | 4.0 | single | i.v. | 0/6 |
| | | | | injection | | |
| | | | | (t = 0) | | |
| 3 | NovoSeven ® | 300 | 4.0 | single | s.c. | 0/6 |
| | | | | injection | | |
| | | | | (t = 0) | | |
| 4 | rVIIa-FP | 300 | 4.0 | single | s.c. | 0/6 |
| | | | | injection | | |
| | | | | (t = 0) | | |

TABLE 3
Time course of FVII: Ag plasma levels
(plasma pool; n = 3/timepoint)

| | | Treatment/Plasma concentration (ng/mL) | | | |
|---|---|---|---|---|---|
| | | NovoSeven ® | rVIIa-FP | NovoSeven ® | rVIIa-FP |
| Timepoint | | i.v. | i.v. | s.c. | s.c. |
| 5 | min | 3346.0 | 5245.5 | | |
| 15 | min | 2220.2 | 4149.7 | | |
| 30 | min | 1718.3 | 4082.7 | <25 | <25 |
| 60 | min. | 1030.1 | 3683.4 | <25 | <25 |
| 90 | min. | 619.0 | 3034.5 | <25 | <25 |
| 2 | h | 364.8 | 2697.7 | <25 | 45.6 |
| 4 | h | 38.3 | 1942.5 | <25 | 49.1 |
| 8 | h | <25 | 726.1 | <25 | 137.3 |
| 24 | h | <25 | 56.7 | <25 | 75.2 |
| 48 | h | <25 | <25 | <25 | <25 |

NovoSeven® was injected at the same dose as rFVIIa-FP and no plasma level was found. It was therefore surprising to find that subcutaneous treatment with rVIIa-FP resulted in a well detectable FVIIa plasma level, with a peak concentration observed after a rather short lag phase following injection (at about 8 hours) and long lasting decay, i.e. for at least 1-2 days. This even outlasted the timecourse of rVIIa-FP following i.v. injection. It had been expected that the lower molecular weight of NovoSeven®, about 50% of the rVIIa-FP, would favor its resorption from the subcutaneous compartment. The results of the study demonstrate that the opposite occurred. The relative bioavailability of subcutaneously administered rVIIa-FP reached about 10%, with no plasma level detected following subcutaneous injection of NovoSeven®.

To judge whether there is a specific benefit of the rVIIa-FP with regard to its resorption or transport to the circulation, comparing the relative bioavailability of rVIIa-FP and NovoSeven® may not be appropriate, because the AUC of s.c. applied rVIIa-FP may be dominated by its long terminal half life, resulting from the continuous release of the rVIIa-FP from the subcutaneous compartment to circulation. In comparison to NovoSeven® the terminal half life following i.v. infusion was already increased more than 6 fold. In addition, the elimination characteristics of rVIIa-FP and NovoSeven® may be differentially impacted by their transport or diffusion from subcutaneous space to circulation. Specific conclusions on this topic may therefore be more reliably based on an assessment of the peak concentrations. For NovoSeven®, plasma concentration never reached the detection limit of 25 ng/mL, while it was about 140 ng/mL for the fusion protein, soon following injection.

As a first step it is possible to estimate, which plasma concentration would be expected following treatment with NovoSeven®, under the assumption that its resorption or transport to the circulation is identical to rVIIa-FP. Correcting for the 60% higher recovery of rVIIa-FP upon i.v. injection, one may expect a peak plasma level of approximately 90 ng/mL. This is clearly above the limit of detection thus excludes a methodological problem underlying, which potentially could have prevented the observation of FVII in plasma, following s.c. treatment with NovoSeven®. In a second step to estimate the minimal benefit achieved by the fusion to albumin, one may assume the best case scenario substantially in favour of NovoSeven®. This would base on the assumption that NovoSeven® actually is transported to plasma but could not be observed because the peak concentration achieved remained just minimally below the detection level of 25 ng/mL at all timepoints. With regard to the duration NovoSeven® may be present in plasma, the best case for NovoSeven® would be to assume an identical time course as rVIIa-FP, despite its terminal half life is about 6 fold shorter. The resulting AUDC for NovoSeven® is 550 h*ng/mL and about 2200 h*ng/mL for rVIIa-FP. It is therefore concluded that in a scenario, highly in favour of NovoSeven®, the in vivo recovery of rVIIa-FP following extravascular injection is at least about 4 fold higher than for NovoSeven®.

### Example 2

### Assessment of Bioavailability of s.c. Applied rIX-FP in a Hemophilia B Model (FIX ko Mice)

To assess, whether extravascular injections might be an option for an improved therapy with rIX-FP (human) a typical representative for an extravascular therapy, subcutaneous injection was chosen. The design of a non-clinical pharmacokinetic study examining such an example is detailed in Table 4. The time course of plasma levels was determined following a single intravenous/subcutaneous injection with 610 IU/kg Berinin® or rIX-FP to a hemophilia B model. rIX-FP was produced according to WO 2007/144173 as described in examples 1 to 3 (pFIX-1088). Corresponding groups were treated with the same dose of FIX:clotting activity. FIX knockout (ko) mice weighing about 25 g were used as a Hemophilia B model. These mice lack the promoter region of the FIX gene thus do not express FIX (Lin et. al. 1997, Blood, 90, 3962-3966). This allowed the analysis of FIX levels following treatment by quantification of FIX activity in the plasma of the ko mice, i.e. functionally active protein. Under short term anesthesia, blood samples were drawn retroorbitally, anticoagulated using calcium citrate to 10 to 20% citrate blood, processed to plasma and stored at −20° C. for the determination of FIX activity. Sampling time points are detailed in table 5. Quantification of FIX activity in plasma was performed by a standard, aPTT based approach (Behring Coagulation Timer). The animals were kept at standard housing conditions.

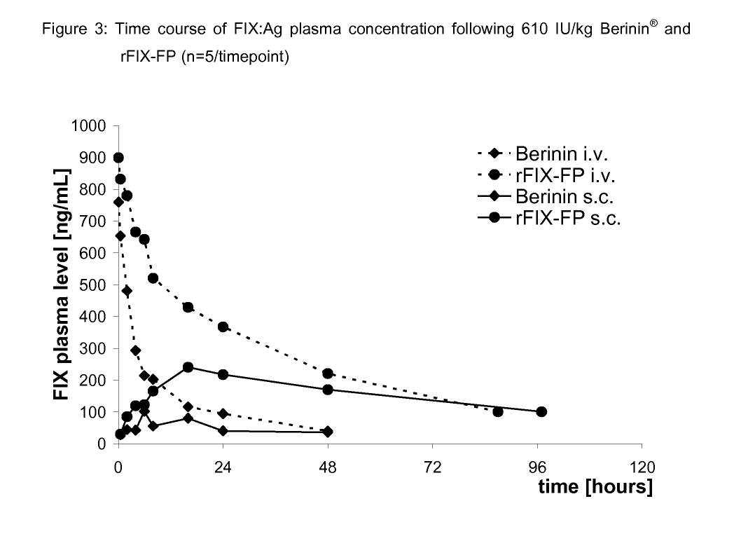

Subcutaneous injection of 610 U/kg Berinin® to FIX ko mice resulted in a small increase of FIX activity in plasma level as compared to the plasma level achieved by an intravenous injection (FIG. 3). Bioavailability following s.c. administration was about 25%. Following s.c. injection FIX could be detected for about 1-2 days. The outcome for the rFIX-FP was clearly different in several aspects:

1) The peak concentration following sc injection of rFIX-FP was about twice as high as seen for Berinin, which is unfused wild type FIX.
2) The bioavailability following s.c. treatment with rFIX-FP was almost 2 fold higher than observed for Berinin (25% vs 45%).
3) In contrast to Berinin the plasma level achieved with s.c. rFIX-FP even exceeded the plasma level achieved with i.v. rFIX-FP in the late phase.

Taken together these results demonstrate that extravascular injections are a valuable option for an improved therapy with rIX-FP. The higher peak concentration opens the possibility for not only prophylactic but possibly even acute substitution therapy in case of a bleeding event. The higher bioavailability allows the application of lower doses and injection volume, increasing cost efficiency and improving tolerability and safety for patients. Finally in a prophylactic setting s.c. application will even allow to increase treatment intervals, because the trough level is reached at a later time point than following i.v. injection.

TABLE 4
Treatment groups

| | | | FIX: | | | | |
|---|---|---|---|---|---|---|---|
| | | | Clotting | | | | |
| | | Mouse | Dose | volume | | | |
| No. | Treatment | Strain | [IU/kg] | [mL/kg] | schedule | route | N |
| 1 | Berinin ® | FIX ko | 610 | 10 | single | i.v. | 25 |
| | | | | | injection | | |
| | | | | | (t = 0) | | |
| 2 | rIX-FP | FIX ko | 610 | 10 | single | i.v. | 25 |
| | | | | | injection | | |
| | | | | | (t = 0) | | |
| 3 | Berinin ® | FIX ko | 610 | 10 | single | s.c. | 20 |
| | | | | | injection | | |
| | | | | | (t = 0) | | |
| 4 | rIX-FP | FIX ko | 610 | 10 | single | s.c. | 20 |
| | | | | | injection | | |
| | | | | | (t = 0) | | |

TABLE 5
Time course of FIX: Clotting plasma levels after intravenous/subcutaneous administration
of 610 IU/kg rIX-FP and Berinin ® (mean ± SD, n = 5/time point)

| | Treatment/Plasma concentration (ng/mL) | | | | |
|---|---|---|---|---|---|
| Timepoint | isotonic | Berinin ® | rIX-FP | Berinin ® | rIX-FP |
| (hours) | saline | 610 IU/kg i.v. | 610 IU/kg i.v. | 610 IU/kg s.c. | 610 IU/kg s.c. |
| 0.083 | 16.1 ± 1.6 | 759.4 ± 8.0 | 898.8 ± 50.9 | — | — |
| 0.5 | — | 652.8 ± 49.9 | 831.1 ± 60.9 | 27.8 ± 4.5 | 29.7 ± 12.0 |
| 2 | — | 480.2 ± 36.4 | 779.6 ± 53.3 | 44.1 ± 6.1 | 84.6 ± 17.8 |
| 4 | — | 292.4 ± 39.5 | 664.8 ± 37.5 | 42.6 ± 10.6 | 119.1 ± 21.0 |
| 6 | — | 213.7 ± 23.3 | 642.0 ± 60.5 | 101.5 ± 21.7 | 122.1 ± 27.1 |
| 8 | — | 202.1 ± 10.3 | 520.1 ± 55.2 | 55.0 ± 7.2 | 165.1 ± 25.3 |
| 16 | — | 115.8 ± 8.5 | 428.1 ± 18.0 | 79.9 ± 14.0 | 239.9 ± 44.5 |
| 24 | — | 94.2 ± 12.5 | 366.9 ± 36.1 | 40.2 ± 7.4 | 216.7 ± 17.8 |
| 48 | — | 39.8 ± 3.5 | 220.2 ± 26.1 | 35.7 ± 2.4 | 169.5 ± 17.6 |
| AUC | — | 63.3 | 282 | 21.6 | 164 |
| (IU h/mL) | | | | | |

### Example 3

### Assessment of Bioavailability of s.c. Applied rVIII-FP in a Hemophilia A Model (FVIII ko Mice)

To assess, whether extravascular injections might be an option for an improved therapy with rVIII-FP (human) a typical representative for an extravascular therapy, subcutaneous injection is chosen. The design of a possible non-clinical pharmacokinetic study performed is detailed in table 7. The timecourse of plasma levels is determined following a single intravenous/subcutaneous injection with ReFacto or rFVIII-FP to a hemophilia A model. Corresponding groups are treated with the same dose of FVIII:clotting activity. FVIII knockout (ko) mice weighing about 25 g are used as a Hemophilia A model. These mice lack exons 16 and 17 and thus do not express FVIII (Bi L. et al, Nature genetics, 1995, Vol 10(1), 119-121; Bi L. et al, Blood, 1996, Vol 88(9), 3446-3450). This allows the analysis of FVIII levels following treatment by quantification of FVIII activity in the plasma of the ko mice. A possible outline of treatment details is provided in table 8. Under short term anesthesia, blood samples are drawn retroorbitally, anticoagulated using calcium citrate to 10 to 20% citrate blood, processed to plasma and stored at −20° C. for the determination of FVIII activity. A possible outline for sampling timepoints is detailed in table 7. Quantification of FVIII activity in plasma is performed by a standard, aPTT based approach (Behring Coagulation Timer). The animals are kept at standard housing conditions.

Subcutaneous injection of 200 U/kg ReFacto to FVIII ko mice results in a small increase of FVIII activity in plasma level as compared to the plasma level expected following an intravenous injection. The comparison with the corresponding treatment with rVIII-FP (human) (groups 2 and 4), generates results for the assessment whether extravascular injections are an option for an improved therapy with rVIII-FP.

TABLE 6
Treatment groups

| | | | FVIII: | | | | |
|---|---|---|---|---|---|---|---|
| | | | Clotting | | | | |
| | | Mouse | Dose | volume | | | |
| No. | Treatment | Strain | [IU/kg] | [mL/kg] | schedule | route | N |
| 1 | ReFacto | FVIII ko | 200 | 10 | single | i.v. | 35 |
| | | | | | injection | | |
| | | | | | (t = 0) | | |
| 2 | rVIII-FP | FVIII ko | 200 | 10 | single | i.v. | 35 |
| | | | | | injection | | |
| | | | | | (t = 0) | | |
| 3 | ReFacto | FVIII ko | 200 | 10 | single | s.c. | 35 |
| | | | | | injection | | |
| | | | | | (t = 0) | | |
| 4 | rVIII-FP | FVIII ko | 200 | 10 | single | s.c. | 35 |
| | | | | | injection | | |
| | | | | | (t = 0) | | |

TABLE 7
Possible outline of time course determination of FVIII: Clotting
plasma levels (mean ± SD, n = 3-5/timepoint)

Treatment/Plasma concentration (U/mL)

| | | ReFacto | rVIII-FP | ReFacto | rVIII-FP |
|---|---|---|---|---|---|
| | | i.v. | i.v. | s.c. | s.c. |
| Timepoint | | 200 U/kg | 200 U/kg | 200 U/kg | 200 U/kg |
| 0 | | | | | |
| 30 | min | | | | |
| 2 | h | | | | |
| 4 | h | | | | |
| 6 | h | | | | |
| 8 | h | | | | |
| 16 | h | | | | |
| 24 | h | | | | |

### Example 4

### Assessment of Bioavailability of s.c. Applied rVWF-FP in a VWD or Hemophilia A Model (VWF or FVIII ko Mice)

To assess, whether extravascular injections might be an option for an improved therapy with rVWF-FP (human) a typical representative for an extravascular therapy, subcutaneous injection is chosen. A possible design of the non-clinical pharmacokinetic study performed is detailed in table 10. Groups 1, 2 and 4 to 6 are treated as detailed in this table, if appropriate other doses of VWF:Ag are injected. The timecourse of plasma levels was determined following a single intravenous/subcutaneous injection with Haemate® P, rVWF-FP or a glycosilation mutant of VWF to a hemophilia A or von Willebrand disease (VWD) model animal. Corresponding groups are treated with the same dose of VWF:Ag. FVIII knockout (ko) mice weighing about 25 g were used as a Hemophilia A model. These mice lack exons 16 and 17 and thus do not express FVIII (Bi L. et al, Nature genetics, 1995, Vol 10(1), 119-121; Bi L. et al, Blood, 1996, Vol 88(9), 3446-3450). VWF ko mice weighing about 25 g are used as a VWD model. These mice lack Exons 4 and 5 of the VWF gene and accordingly do not express VWF (Denis C. et al, Proc. Natl. Acad. Sci. USA, 1998, Vol 95, 9524-9529).

Treatment details are provided in table 6. Under short term anesthesia, blood samples are drawn retroorbitally, anticoagulated using calcium citrate to 10 to 20% citrate blood, processed to plasma and stored at −20° C. for the determination of FVIII activity. Sampling timepoints are detailed in table 7. Quantification of human VWF:Ag in plasma is performed by a commercially available ELISA testkit (Asserachrom®, Diagnostica Stago). The animals are kept at standard housing conditions.

Subcutaneous injection of about 1400 U/kg VWF:Ag U/kg Haemate® P to mice results in an increase of human VWF:Ag in plasma as compared to the plasma level expected following an intravenous injection. The comparison with the corresponding treatment with rVWF-FP (human) (groups 2 and 5), as well as the corresponding treatment with VWF glycosilation mutants (groups 3 and 6) generates results for the assessment whether extravascular injections are an option for an improved therapy with rVWF-FP and VWF glycosilation mutants.

TABLE 8
Possible treatment groups

| | | | VWF: Ag | | | | |
|---|---|---|---|---|---|---|---|
| | | Mouse | Dose | volume | | | |
| No. | Treatment | Strain | [IU/kg] | [mL/kg] | schedule | route | N |
| 1 | Haemate ® P | FVIII/VWF | 1457 | 10 | single | i.v. | 20 |
| | | ko | | | injection | | |
| | | | | | (t = 0) | | |
| 2 | rVWF-FP | FVIII/VWF | 1457 | 10 | single | i.v. | 20 |
| | | ko | | | injection | | |
| | | | | | (t = 0) | | |
| 3 | VWF | FVIII/VWF | 1457 | 10 | single | i.v. | 20 |
| | Glycosilatio | ko | | | injection | | |
| | | | | | (t = 0) | | |
| 4 | Haemate ® P | FVIII/VWF | 1457 | 10 | single | s.c. | 20 |
| | | ko | | | injection | | |
| | | | | | (t = 0) | | |
| 5 | rVWF-FP | FVIII/VWF | 1457 | 10 | single | s.c. | 20 |
| | | ko | | | injection | | |
| | | | | | (t = 0) | | |
| 6 | VWF | FVIII/VWF | 1457 | 10 | single | s.c. | 20 |
| | Glycosilatio | ko | | | injection | | |
| | | | | | (t = 0) | | |

TABLE 9
Possible outline for the determination of VWF: Ag plasma levels
(% of the norm) following a single s.c. injection of 500 U/kg
FVIII: C of different VWF Haemate ® P preparations to FVIII ko
mice (mean ± SD; n = 5 per timepoint)
Treatment/Plasma concentration (U/mL)

| | | | | VWF | | | VWF |
|---|---|---|---|---|---|---|---|
| | | | rVWF- | Glyco | | rVWF- | Glyco |
| | | Haemate ® P | FP | mutant. | Haemate ® P | FP | mutant. |
| Time | | i.v. | i.v. | i.v. | s.c. | s.c. | s.c. |
| baselin | | | | | | | |
| 2 | h | | | | | | |
| 4 | h | | | | | | |
| 8 | h | | | | | | |
| 16 | h | | | | | | |

1-17. (canceled)

18. A method of treating a bleeding disorder comprising administering by a non-intravenous route to a subject in need thereof a therapeutically effective dose of a pharmaceutical preparation comprising an albumin-fused coagulation factor, thereby treating the bleeding disorder.

19. The method according to claim 18, wherein the coagulation factor is Factor IX, Factor VII, Factor VIII, von Willebrand Factor, Factor V, Factor X, Factor XI, Factor XII, Factor XIII, Factor I, Factor II (Prothrombin), Protein C, Protein S, GAS6, Protein Z, or an activated form thereof.

20. The method according to claim 19, wherein the coagulation factor is Factor IX, Factor VII, Factor VIII, von Willebrand Factor, or an activated form thereof.

21. The method according to claim 18, wherein the bleeding disorder is familial or acquired hemophilia A or B; trauma; bleeding during a surgical procedure; intracerebral haemorrhage; subarachnoid haemorrhage; sub- or epidural bleeding; bleeding due to blood loss and hemodilution; bleeding due to disseminated intravascular coagulation (DIC); bleeding due to liver cirrhosis, liver dysfunction, fulminant liver failure, or liver biopsy; bleeding after organ transplantation; bleeding from gastric varices or peptic ulcer; gynaecological bleeding; bleeding associated with burns; bleeding associated with amyloidosis; hematopoietic stem cell transplantation associated with platelet disorder; bleeding associated with malignancies; bleeding associated with infection with a haemorrhaging virus, or bleeding associated with pancreatitis.

22. The method according to claim 21, wherein the gynaecological bleeding is dysfunctional uterine bleeding (DUB), bleeding due to premature detachment of the placenta, periventricular haemorrhage in low birth weight children, post partum haemorrhage, or fatal distress of newborns.

23. The method according to claim 18, wherein the non-intravenous administration is subcutaneous, transdermal, or intramuscular administration.

24. The method according to claim 23, wherein the non-intravenous administration is subcutaneous administration.

25. The method according to claim 18, wherein the coagulation factor is connected to albumin via a peptidic linker.

26. The method according to claim 25, wherein the peptidic linker is proteolytically cleavable.

27. The method according to claim 18, wherein the coagulation factor is Factor IX.

28. The method according to claim 27, wherein the bleeding disorder is hemophilia B.

29. The method according to claim 18, wherein the coagulation factor is Factor VIIa.

30. The method according to claim 29, wherein the bleeding disorder is hemophilia A or B.

31. The method according to claim 18, wherein the coagulation factor is Factor VIII.

32. The method according to claim 31, wherein the bleeding disorder is hemophilia A.

33. The method according to claim 18, wherein the coagulation factor is von Willebrand Factor.

34. The method according to claim 33, wherein the bleeding disorder is von Willebrand's disease.

35. A method of treating a bleeding disorder comprising subcutaneously administering to a subject in need thereof a therapeutically effective dose of a pharmaceutical preparation comprising an albumin-fused Factor VIIa, thereby treating the bleeding disorder.

36. The method of claim 35, wherein the bleeding disorder is hemophilia A or hemophilia B.

37. A method of treating a bleeding disorder comprising subcutaneously administering to a subject in need thereof a therapeutically effective dose of a pharmaceutical preparation comprising an albumin-fused Factor IX, thereby treating the bleeding disorder.

38. The method of claim 37, wherein the bleeding disorder is hemophilia B.
